(19) United States
(12) Patent Application Publication
(10) Pub. No.: US 2010/0233218 A1
(43) Pub. Date: Sep. 16, 2010

(54) COMBINATION ENZYME FOR CYSTIC FIBROSIS

(75) Inventor: Joan M. Fallon, Bronxville, NY (US)

Correspondence Address: FISH & RICHARDSON P.C., PO BOX 1022, MINNEAPOLIS, MN, 55440-1022, US

(73) Assignee: CUREMARK LLC, Rye, NY (US)

(21) Appl. No.: 12/786,739

(22) Filed: May 25, 2010

Related U.S. Application Data
(63) Continuation of application No. 11/232,180, filed on Sep. 21, 2005, now abandoned.
(60) Provisional application No. 60/613,666, filed on Sep. 28, 2004.

(51) Int. Cl. A61K 9/00; A61K 38/43; A61K 38/46; A61K 38/47; A61K 38/48; A61P 1/18

(52) U.S. Cl. 424/400; 424/94.1; 424/94.6; 424/94.61; 424/94.65; 424/94.64

(57) ABSTRACT

A stable preparation of digestive/pancreatic enzymes which can be readily formed into a dosage formulation is provided as a treatment of pancreatic insufficiency in persons having cystic fibrosis. The dosage formulation can be administered either by an oral preparation including, but not limited to, a microcapsule, mini-capsule, time released capsule, sprinkle or other methodology. A further object of this invention is to provide a stabilized preparation of a combination medicant which resists degradation by light, heat, humidity or association with commonly used excipients.

### CROSS REFERENCE TO RELATED APPLICATION

This application is a continuation of U.S. patent application Ser. No. 11/232,180, filed Sep. 21, 2005, which claims priority under 35 USC §119(e) to U.S. Patent Application Ser. No. 60/613,666, filed on Sep. 28, 2004, the entire contents of which are hereby incorporated by reference.

### FIELD OF THE INVENTION

The present invention is directed to therapeutic agents for the treatment of pancreatic insufficiency in those with cystic fibrosis and other pancreatic disorders. More specifically, the present invention relates to stable pharmaceutical preparations containing but not limited to digestive and/or pancreatic enzymes including but not limited to amylases, proteases, cellulase, papaya, bromelain, lipases, chymotrypsin, pancreatin and pancrelipase. This combination is made either by direct compression, wet granulation or other methods including but not limited to the use of Prosolv technology, and/or time-release technology. The invention further relates to novel combinations of these enzymes heretofore not previously utilized in the population with cystic fibrosis or other pancreatic insufficiencies.

### BACKGROUND OF THE INVENTION

Cystic fibrosis (CF) is one of the most common fatal genetic disorders. If affects the lungs and digestive systems of children and adults with the disease preventing adequate enzymatic digestion of food, as well as difficult breathing associated with thick mucous secretions in the lungs. The lack of proper absorption of nutrients in this population due to improper release of digestive enzymes from the pancreas. Without proper digestion of foodstuffs by enzymatic breakdown will allow for a dearth of necessary nutrients for the child/adult with CF.

At present those with CF must consume a large number of enzymes (on average 20 pills or more a day) with every meal to help them absorb adequate nutrition from their food. This large number of pills is cumbersome for those CF, and also lends itself to underutilization of the enzymes and a lack of proper nutrition for those with this disease.

It is estimated that CF occurs in 1 in 2,500 to 1 in 3,000 live births. The occurrence is most common in Caucasian children.

It is known that presently marketed pharmaceutical preparations containing digestive/pancreatic enzymes utilized by CF and others with pancreatic insufficiency are known to exhibit deficiencies with regard to content uniformity, stability and shelf life. In April of 2004 the US Food and Drug Administration issued a guideline as to the filing of new drug applications for these preparations as the presently marketed preparations of the digestive/pancreatic enzyme formulations were deemed inadequate. More specifically since digestive/pancreatic enzymes can degrade rapidly under conditions of high humidity or in the presence of other moisture sources, under light and under conditions of high temperature and extremes in pH. The present enzymes on the market have been deemed inadequate. Moreover, digestive enzymes are known to degrade certain pharmaceutical excipients such as carbohydrates, including lactose, sucrose, dextrose and starch, as well as certain dyes making the current compounds on the market substandard and potentially under medicating those who need the enzymes.

### SUMMARY OF THE INVENTION

A goal of the present invention is to provide a stable preparation of digestive/pancreatic enzymes which can be readily formed into a dosage formulation. While well known in the art that CF patients require digestive/pancreatic enzymes, a novel formulation and dosing is proposed here which heretofore has not been utilized in CF patients. The dosage formulation can be administered either by an oral preparation including but not limited to a microcapsule, minicapsule, time released capsule or other methodology. A further object of this invention is to provide a stabilized preparation of a combination medicant which resists degradation by light, heat, humidity or association with commonly used excipients.

A further goal of the invention is to provide a pharmaceutical preparation in which an excipient provides a matrix to capture and protect the product before delivery. Another goal of the invention is to provide a novel pharmaceutical preparation whereby the individual who takes the preparation has a reduction in the number of capsules/tablets per dosage.

There is provided by the present invention a stabilized pharmaceutical preparation comprising a therapeutically effective amount of a protease, and amylase and a lipase. Further the invention will be in the form of a tablet, capsule, or time released formula of the same to reduce the amount of pills/tablets/capsules and/or sprinkles per dosage. The preparation of the present invention provides a stabilizing matrix consisting essentially of but not limited to a solidified microcrystalline cellulose which captures and protects therapeutically effective amounts of digestive enzyme particles within the stabilizing matrix known in the art as the PROSOLV® technology.

These and other aspects, features and advantages of the present invention will be described and become apparent from the following description of the preferred embodiments.

### BRIEF DESCRIPTION OF THE DRAWINGS

FIG. 1 is a list of the potential various combinations of digestive/pancreatic enzymes of the present invention.

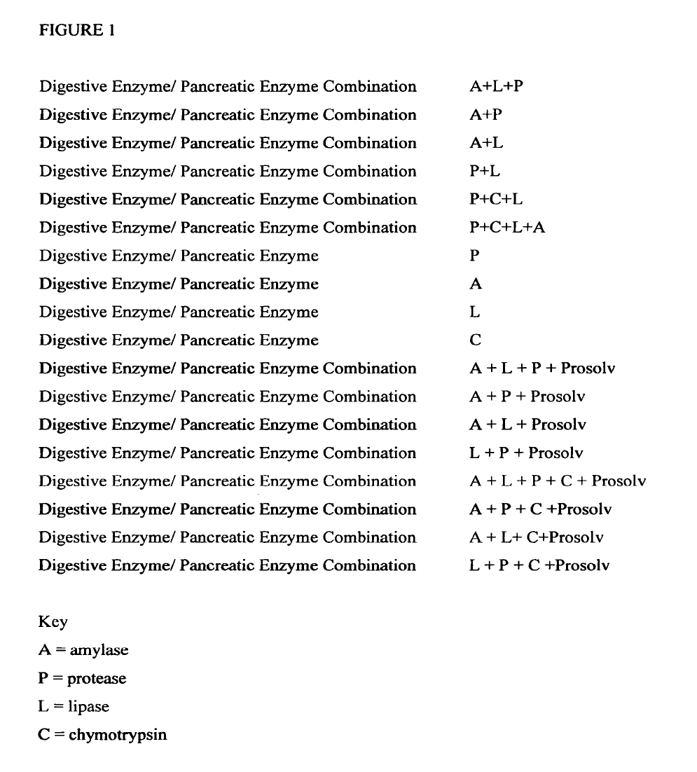

### DETAILED DESCRIPTION OF THE PREFERRED EMBODIMENTS

The present invention is to provide a stable preparation of digestive/pancreatic enzymes which can be readily formed into a dosage formulation. The dosage formulation can be administered either by an oral preparation including but not limited to a microcapsule, mini capsule, time released capsule, sprinkle or other methodology. The invention is designed to provide a stabilized preparation of a combination medicant which resists degradation by light, heat, humidity or association with commonly used excipients.

While it is well known to one skilled in the art that digestive/pancreatic enzymes have been utilized by those with CF and those with pancreatic insufficiency, this novel combination of enzymes as well as the method of production has not been heretofore utilized by this population.

The invention is designed to provide a pharmaceutical preparation in which an excipient provides a matrix to capture and protect the product before delivery. Another goal of the invention is to provide a novel pharmaceutical preparation whereby the individual who takes the preparation has a reduction in the number of capsules/tablets per dosage. There is provided by the present invention a stabilized pharmaceutical preparation comprising a therapeutically effective amount of a protease, and amylase and a lipase. Further the invention will be in the form of a tablets, capsules, time released tablets or capsules, sprinkles or other form to reduce the amount of pills/tablets/capsules sprinkles per dosage. The preparation of the present invention provides a stabilizing matrix consisting essentially of but not limited to a solidified microcrystalline cellulose which captures and protects therapeutically effective amounts of digestive enzyme particles within the stabilizing matrix. This can be done through the use of what is known in the art as PROSOLV® technology.

In one embodiment, the present invention is directed to a direct compression method for the manufacture of a pharmaceutical tablet preparation comprising the steps of: (a) forming an active blend by blending an intimate admixture silicified microcrystalline cellulose and a therapeutic agent comprising one or more digestive enzymes (b) forming a color blend by blending an intimate admixture one or more pharmaceutically acceptable dyes and silicified microcrystalline cellulose if color is necessary; (c) combining the active blend, the color blend and a disintegrant into a preblend; (d) adding a lubricant to the preblend to form final blend; and (e) compressing the final blend to form a pharmaceutical tablet preparation or a mixture of time released microtabs or a time released tablet.

This invention is accomplished by combining the digestive enzymes with one of the patented PROSOLV® technologies, i.e.: PROSOLV SMCC® 50 (silicified microcrystalline cellulose) or PROSOLV SMCC® 90 (silicified microcrystalline cellulose), or other PROSOLV®technologies. When employing the PROSOLV® method, the silicified microcrystalline cellulose (SMCC) used in the preparation of the present invention may be any commercially available combination of microcrystalline cellulose granulated with colloidal silicon dioxide. The SMCC generally will be as described in Sherwood et al, Pharm. Tech., October 1998, 78-88, and U.S. Pat. No. 5,585,115, incorporated herein by reference in its entirety. SMCC can be obtained commercially from Edward Mendell Company, Inc., a subsidiary of Penwest Ltd., under the name PROSOLV SMCC®. There are different grades of SMCC available, with particle size being the differentiating property among the grades. For example, PROSOLV SMCC® 90 has a median particle size, by sieve analysis, in the region of 90 micrometers. PROSOLV SMCC® 50 has a median particle size, by sieve analysis, in the region of about 40-50 micrometers.

The pharmaceutical preparation of the present invention may be prepared using a direct compression method, a dry granulation method or by wet granulation. Preferably, the digestive/pancreatic enzyme preparation of the present invention will be prepared using a direct compression process. Each of these processes consists of two main steps: blending and compression.

The blending step is composed of an active blend, color blend, pre-blend, and final blend (lubrication). The formulation of the present invention may include a number of other ingredients for optimal characteristics of the pharmaceutical composition. Such other ingredients and the amounts to be used are within the knowledge of persons having ordinary skill in the art and are known in the pharmaceutical art. These may include disintegrates, lubricants and/or coloring agents, among others. Suitable disintegrants include, for example, sodium starch glycolate, other starches such as pregelatinized starch, and celluloses. Suitable lubricants may be provided such as magnesium stearate, calcium stearate, talc and stearic acid. Any coloring agent certified by the FDA may be used, such as FD&C Yellow #6, among others.

PROSOLV® is a combination of excipients which allow for optimized flow, compaction and product uniformity. This technology allows for uniformity in this combination, as well as manufacturing a very small tablet which would be amenable for children. With PROSOLV® technology, the ingredients are not just blended, but are co processed, which assures that equal particles are uniformly distributed and these results are easily reproducible. This allows for stability and superb product quality.

Whether utilizing the PROSOLV® method or other methodology, the medicant will be formulated such and manufactured such that there the particles will be uniformly distributed and there will be no overage with respect to the amount of enzyme found in the preparation. Said new drug formulation can be found in but is not limited to formulations which include digestive/pancreatic enzymes with and without the utilization of the PROSOLV® technology.

The digestive/pancreatic enzyme combination component of the overall combination may include but are not limited to one or more of the following: amylases, proteases, cellulase, papaya, bromelain, lipases, chymotrypsin, trypsin. These enzymes can be in the form of animal or plant derivatives, natural or synthetic.

Each of these combinations can be made into a pulse dose formulation wherein the time release portion of the tablet can be with the enzyme portion, dosing therefore can be delivered in the tablet or micro pellets in a single pulse delivery or a time release delivery. These combinations are not limited by number or scope of digestive enzymes. This invention is further unique by virtue of the compression and co-processing methodology which the PROSOLV® technology brings to the mixture of medicant and digestive enzyme. The pill size therefore can be significantly reduced, the amount of medicant and digestive enzyme significantly regulated and reproducible, and the novel combination can be delivered either directly through the pill and dissolved by the body, or can be delivered in a pulse dosing fashion which renders the digestive enzymes or its derivatives delivered in a time release fashion.

The PROSOLV® technology further adds improved material flow while maintaining compaction, manufacturing speeds can be improved, and allows for high or low drug loading applications as well as time or pulse release delivery. Further the technology allows for a pill for tablet or micro tablets to be produced which has optimal content uniformity, direct compression without granulation, fewer numbers of excipients and fillers, and a smaller tablet.

The following examples demonstrate the formulations which conform to the above conditions of manufacture with or without utilizing the PROSOLV® technology. It is to be understood that these examples are set forth by way of illustration only, and nothing therein shall be taken as a limitation upon the overall scope of the invention.

### Example 1

The following outlines a formulary for digestive/pancreatic enzymes for CF and other pancreatic insufficiencies:

| Amylase | 10,000-60,000 | USP |
|---|---|---|
| Protease | 10,000-50,000 | USP |
| Lipase | 4,000-20,000 | USP |
| Pancreatin | 2,000-6,000 | USP |
| Chymotrypsin | 2-5 | mg |
| Trypsin | 60-100 | mg |
| Papain | 3,000-10,000 USP units/mg | |
| Papaya | 30-60 | mg |

### Example 2

The following outlines a formulary for digestive/pancreatic enzymes for CF and other pancreatic insufficiencies:

| | |
|---|---|
| Protease | 10,000 |
| Chymotrypsin | 2 mg |
| Trypsin | 60 mg |
| Papaya | 30 mg |

### Example 3

The following outlines a formulatory for digestive/pancreatic enzymes for CF and other pancreatic insufficiencies:

| | |
|---|---|
| Amylase | 20,000 USP units/mg |
| Protease | 30,000 USP units/mg |
| Lipase | 30,000 USP units/mg |

### Example 4

The following outlines a formulatory for digestive/pancreatic enzymes for CF and other pancreatic insufficiencies:

| | | |
|---|---|---|
| Amylase | 30,000 | USP units/mg |
| Protease | 40,000 | USP units/mg |
| Lipase | 30,000 | USP units/mg |
| Chymotrypsin | 2 | mg |

### Example 5

The following outlines a formulatory for digestive/pancreatic enzymes for CF and other pancreatic insufficiencies:

| | | |
|---|---|---|
| Amylase | 30,000 | USP units/mg |
| Protease | 40,000 | USP units/mg |
| Lipase | 30,000 | USP units/mg |
| Chymotrypsin | 2 | mg |
| Papaya | 30 | mg |

### Example 6

The following outlines a formulatory for digestive/pancreatic enzymes for CF and other pancreatic insufficiencies:

| | | |
|---|---|---|
| Amylase | 30,000 | USP units/mg |
| Protease | 40,000 | USP units/mg |
| Lipase | 30,000 | USP units/mg |
| Chymotrypsin | 2 | mg |
| Papain | 6,000 | USP units/mg |

### Example 7

The following outlines a formulatory for digestive/pancreatic enzymes for CF and other pancreatic insufficiencies:

| | | |
|---|---|---|
| Amylase | 30,000 | USP units/mg |
| Protease | 40,000 | USP units/mg |
| Lipase | 30,000 | USP units/mg |
| Chymotrypsin | 2 | mg |
| Papain | 8,000 | USP units/mg |

What is claimed is:

1. A method for treating one or more symptoms of cystic fibrosis in a patient comprising administering a pharmaceutical composition comprising a therapeutically effective amount of a protease, an amylase, and a lipase, and wherein the pharmaceutical preparation comprises microcrystalline cellulose.

2. The method of claim 1, wherein the pharmaceutical composition further comprises one or more of cellulase, bromelain, papain, pancreatin, chymotrypsin, and trypsin.

3. The method of claim 1, wherein the pharmaceutical composition is an oral dosage form.

4. The method of claim 1, wherein the pharmaceutical composition is a tablet, capsule, pellet or sprinkle dosage form.

5. The method of claim 1, wherein the pharmaceutical composition is capable of providing time release delivery of the protease, amylase, and lipase.

6. The method of claim 1, wherein the microcrystalline cellulose is silicified microcrystalline cellulose.

7. The method of claim 6, wherein the silicified microcrystalline cellulose has a mean particle size of about 90 micrometers.

8. The method of claim 6, wherein the silicified microcrystalline cellulose has a mean particle size of about 40-50 micrometers.

9. The method of claim 1, wherein the protease, amylase, and lipase are, independently, derived from animal or plants or are synthetically prepared.

10. The method of claim 1, wherein the pharmaceutical composition further comprises chymotrypsin.

11. The method of claim 1, wherein the pharmaceutical composition further comprises one or more disintegrants, lubricants, or coloring agents.

12. The method of claim 1, wherein the pharmaceutical composition is capable of providing a pulse delivery of the protease, amylase, and lipase.
